(19) United States
(12) Patent Application Publication
(10) Pub. No.: US 2024/0242810 A1
(43) Pub. Date: Jul. 18, 2024

(54) INSULIN ON BOARD FOR GLUCOSE SENSITIVE INSULIN

(71) Applicant: Novo Nordisk A/S, Bagsvaerd (DK)

(72) Inventors: Henrik Bengtsson, Taastrup (DK); Tinna Bjoerk Aradottir, Copenhagen (DK); Sarah Ellinor Engell, Koebenhavn N (DK); Thomas Emil Ryde, Koebenhavn K (DK)

(21) Appl. No.: 18/289,294

(22) PCT Filed: May 5, 2022
(86) PCT No.: PCT/EP2022/062181

§ 371 Date: Nov. 2, 2023

(30) Foreign Application Priority Data
May 5, 2021 (EP) 21172245.9

(51) Int. Cl. G16H 20/17

(52) U.S. Cl. CPC G16H 20/17

(57) ABSTRACT

A method of estimating insulin on board (IOB) for a given glucose sensitive insulin (GSI) in a subject is provided. The method comprises the steps (i) for the given GSI, providing at least one rate constant (r_{c}) as function of glucose concentration (r_{c}(G)), (ii) providing for a period of time a continuous blood glucose log G(t) from the subject, (iii) providing for the period of time 5 an insulin dose log I(t) from the subject, (iv) based on rc(G) and G(t), calculating for each rc a rate constant as function of time (r_{c}(t)), and (v) based on the at least one r_{c}(t) and I(t) and using an estimating algorithm, calculating an estimated IOB for the subject.

The present disclosure generally relates to systems and methods for assisting patients and health care practitioners in managing insulin treatment to diabetics. In a specific aspect the present invention relates to systems and methods suitable for use in a diabetes management system supporting a patient in treatment with a glucose sensitive insulin (GSI).

### BACKGROUND

Diabetes mellitus (DM) is impaired insulin secretion and variable degrees of peripheral insulin resistance leading to hyperglycaemia. Type 2 diabetes mellitus is characterized by progressive disruption of normal physiologic insulin secretion. In healthy individuals, basal insulin secretion by pancreatic B cells occurs continuously to maintain steady glucose levels for extended periods between meals. Also in healthy individuals, there is prandial secretion in which insulin is rapidly released in an initial first-phase spike in response to a meal, followed by prolonged insulin secretion that returns to basal levels after 2-3 hours. Years of poorly controlled hyperglycaemia can lead to multiple health complications. Diabetes mellitus is one of the major causes of premature morbidity and mortality throughout the world.

Effective control of blood/plasma glucose can prevent or delay many of these complications but may not reverse them once established. Hence, achieving good glycaemic control in efforts to prevent diabetes complications is the primary goal in the treatment of type 1 and type 2 diabetes. Smart titrators with adjustable step size and physiological parameter estimation and pre-defined fasting blood glucose target values have been developed to administer insulin medicament treatment regimens.

There are numerous non-insulin treatment options for diabetes, however, as the disease progresses, the most robust response will usually be with insulin. In particular, since diabetes is associated with progressive β-cell loss many patients, especially those with long-standing disease will eventually need to be transitioned to insulin since the degree of hyperglycemia (e.g., HbA1c≥8.5%) makes it unlikely that another drug will be of sufficient benefit.

The ideal insulin regimen aims to mimic the physiological profile of insulin secretion as closely as possible. There are two major components in the insulin profile: a continuous basal secretion and prandial surge after meals. The basal secretion controls overnight and fasting glucose while the prandial surges control postprandial hyperglycemia.

Based on the time of onset and duration of their actions, injectable formulations can be broadly divided into basal (long-acting analogues [e.g., insulin detemir and insulin glargine] and ultralong-acting analogues [e.g., insulin degludec]) and intermediate-acting insulin [e.g., isophane insulin] and prandial (rapid-acting analogues [e.g., insulin aspart, insulin glulisine and insulin lispro]). Premixed insulin formulations incorporate both basal and prandial insulin components.

Glucose sensitive insulin (GSI), also called glucose sensitive insulin (GRI), is a new generation of drugs where insulin action is influenced by the glycaemic level, see e.g. US 2019/247468. The adaptive drug action can be achieved in multiple ways, e.g. by modulating the insulin's potency, concentration, or clearance relative to the glycaemic level. This mechanism mimics the regulatory function of insulin producing beta cells in non-diabetic people. When glycaemic levels drop, the action of a GSI will decrease and thereby reduce the risk of hypoglycaemia. Similarly, a rise in blood glucose concentration will increase the insulin action, reducing the time spent in hyperglycaemia. U.S. Pat. No. 10,398,781 discusses the discovery of specific GSI molecules and the fact that they can modify PK/PD profiles.

In a paper “Rational Design of Glucose-Responsive Insulin Using Pharmacokinetic Modelling” (Bakh et al., 2017, Advanced Healthcare Materials—Wiley Online Library), GSI design parameters are linked to therapeutic efficacy, which can be used for drug design, and the optimal PK/PD parameters for different dose sizes are determined.

There are various recommended insulin regimes, such as (1) multiple injection regimen: rapid-acting insulin before meals with long-acting insulin once or twice daily, (2) premixed analogues or human premixed insulin once or twice daily before meals, and (3) intermediate- or long-acting insulin once or twice daily.

Algorithms can be used to generate recommended insulin dose and treatment advice for diabetes patients. However, as glycaemic levels fluctuate over time, so will the drug usage and the amount of insulin available in the body at a given point in time, the insulin on board (IOB). For traditional insulins, i.e. non-GSIs, the insulin on board is typically calculated from the drug-specific PK/PD curve describing insulin decay over time. The method is not applicable for GSIs as it does not account for the glycaemic influence on the drug's PK/PD profile.

Correspondingly, it is an object of the present invention to provide methods and systems allowing GSI usage over time and thereby IOB to be estimated to ensure that the internal depot of GSI is not exhausted and results in suboptimal treatment.

### DISCLOSURE OF THE INVENTION

In the disclosure of the present invention, embodiments and aspects will be described which will address one or more of the above objects or which will address objects apparent from the below disclosure as well as from the description of exemplary embodiments.

In a first aspect of the invention a method of estimating insulin on board (IOB) for a given glucose sensitive insulin (GSI) in a subject is provided, the method comprising the steps: for the given GSI, providing at least one rate constant (r_{c}) as function of glucose concentration (r_{c}(G)), providing for a period of time a continuous blood glucose log G(t) from the subject, providing for the period of time an insulin dose log I(t) from the subject, based on r_{c}(G) and G(t), calculating for each r_{c} a rate constant as function of time (r_{c}(t)), and based on the at least one r_{c}(t) and I(t) and using an estimating algorithm, calculating an estimated IOB for the subject.

In this way a method is provided allowing GSI usage over time and thereby IOB to be estimated to ensure that the internal depot of GSI is not exhausted and results in suboptimal treatment.

The method of estimating IOB may be based on a compartment model. The compartment model may comprise at least 2 compartments, at least one transfer rate constant between compartments, and at least one clearance rate constant, wherein at least one of the rate constants being a function of glucose concentration. Alternatively a data-driven model may be used.

The method may comprise the further step of providing for the period of time a meal size log M(t) from the subject, this allowing the calculation of IOB for the subject to be additionally based on M(t).

In a further aspect of the invention a computing system for estimating insulin on board (IOB) for a given glucose sensitive insulin (GSI) in a subject is provided. The system comprises one or more processors and a memory, the memory comprising instructions that, when executed by the one or more processors, perform a method responsive to receiving a request for calculation of an IOB value. The method comprises the steps of: for the given GSI, providing at least one rate constant (r_{c}) as function of glucose concentration (r_{c}(G)), providing for a period of time a continuous blood glucose log G(t) from the subject, providing for the period of time an insulin dose log I(t) from the subject, based on r_{c}(G) and G(t), calculating for each r_{c} a rate constant as function of time (r_{c}(t)), and based on the at least one r_{c}(t) and I(t) and, using an estimating algorithm, calculating IOB for the subject.

The method of estimating IOB may be based on a compartment model. The compartment model may comprise at least 2 compartments, at least one transfer rate constant between compartments, and at least one clearance rate constant, wherein at least one of the rate constants being a function of glucose concentration. Alternatively a data-driven model may be used.

The method may comprise the further step of: providing for the period of time a meal size log M(t) from the subject, wherein the calculation of the estimated IOB for the subject is additionally based on M(t).

The above-described computing system may be adapted to provide a long-acting or ultra-long-acting insulin dose recommendation (ADR) for a subject to treat diabetes mellitus, the memory further comprising: instructions that, when executed by the one or more processors, perform a method responsive to receiving a dose guidance request (DGR), the method providing the long-acting or ultra-long-acting insulin ADR, wherein the recommendation is being calculated based on the estimated IOB.

### BRIEF DESCRIPTION OF THE DRAWINGS

In the following embodiments of the invention will be described with reference to the drawings, wherein

FIG. 1A shows a compartment model in which insulin absorption depends on glucose concentration,

FIG. 1B shows a compartment model in which insulin clearance depends on glucose concentration,

FIG. 1C shows a compartment model in which insulin clearance depends on a combination of several mechanisms,

FIG. 2 shows an example of a rate constant as function of glucose concentration,

FIG. 3 shows an algorithm overview for calculation of an IOB value,

FIG. 4 shows the sensitivity function as a function of BG concentration for GSI respectively normal basal insulin, and

FIG. 5 shows for 3 different cases BG values, basal insulin dosing, meal events, IOB and the sensitivity function as a function of time.

### DESCRIPTION OF EXEMPLARY EMBODIMENTS

The present invention relates to an algorithm adapted to estimate the insulin on board (IOB) using continuous glucose measurements and insulin injection data as input. The algorithm may be based on a compartment model of drug-action with one or more glucose-dependent rate constants describing drug usage. The inputs are used to determine how the rate constant changes over time in relation to the glucose concentration. Based on the dynamic calculation of the rate constant, the algorithm estimates the drug usage and calculates the IOB.

The algorithm of the present invention may be used as a stand-alone solution providing a user with information about his/her IOB, however, the algorithm may also be used as part of an overall diabetes dose guidance system that helps people with diabetes by generating recommended insulin doses based on calculated IOB values.

In such a system a given algorithm is used to generate recommended insulin doses and treatment advice for diabetes patients based on BG data, insulin dosing history and, in more advanced applications, other factors like meals, physical activity, stress, illness etc. may be taken into consideration.

Essentially such a system comprises a back-end engine (“the engine”) used in combination with an interacting systems in the form of a client and an operating system. The client from the engine's perspective is the software component that requests dose guidance. The client gathers the necessary data (e.g. CGM data, insulin dose data, patient parameters) and requests dose guidance from the engine. The client then receives the response from the engine.

On a small local scale the engine may run directly as an app on a given user's smartphone and thus be a self-contained application comprising both the client and the engine. Alternatively, the system setup may be designed to be implemented as a back-end engine adapted to be used as part of a cloud-based large-scale diabetes management system. Such a cloud-based system would allow the engine to always be up-to-date (in contrast to app-based systems running entirely on e.g. the patient's smartphone), would allow advanced methods such as machine learning and artificial intelligence to be implemented, and would allow data to be used in combination with other services in a greater “digital health” set-up. Such a cloud-based system ideally would handle a large amount of patient requests for dose recommendations.

Although a “complete” engine may be designed to be responsible for all computing aspects, it may be desirable to divide the engine into a local and a cloud version to allow the patient-near day-to-day part of the dose guidance system to run independently of any reliance upon cloud computing. For example, when the user via the client app makes a request for dose guidance the request is transmitted to the engine which will return a dose recommendation. In case cloud access is not available the client app would run a dose-recommendation calculation using local data. Dependent upon the user's app-settings the user may or may not be informed.

An aim of the present invention is to calculate IOB for glucose sensitive insulin (GSI) where the IOB cannot be calculated as a time decay as the drug usage is glucose dependent.

It is assumed that a drug-specific model that describes how the pharmacokinetics and pharmacodynamics (PK/PD) of the GSI change as a function of the glucose concentration is provided. The GSI action is delayed such that a rise in glucose levels is visible and can be used to estimate drug usage. Inputs for such a model would be (1) continuous glucose monitor data, and (2) insulin injection data.

In a first embodiment to estimate the insulin on board, the algorithm uses a drug-specific compartment model where one or more rate constants are glucose-dependent. The affected rate constants may e.g. be the absorption rate(s), the activation rate (if insulin has an active and inactive state), the glucose-independent clearance rate, or the glucose dependent clearance rate. A known, drug-specific correlation between glucose concentration and rate constants is used to estimate the insulin on board.

A compartment model is an example of how the speed the insulin moves from injection site to clearance can be modelled. Alternatively a data-driven model may be used. A compartment model is a model where each state typically represents the physical location that the insulin is in. In the present example the depot state is the site where the drug is deposited. The insulin then moves at some rate to the transit state. Insulin arrives in the transit state at some rate but also continuous onto the central state at another rate. The central state models that the insulin has arrived at tissues or organs. The rate constants depend on how fast insulin moves from one compartment to the other, e.g. how fast insulin moves from the injection site to the blood. For a GSI the rates are not constant but vary depending on glucose concentration, however, in the exemplary model the rate constant is fixed to a given value (e.g. mean value for all patients at a given BG) which is then multiplied by a factor termed the sensitivity function.

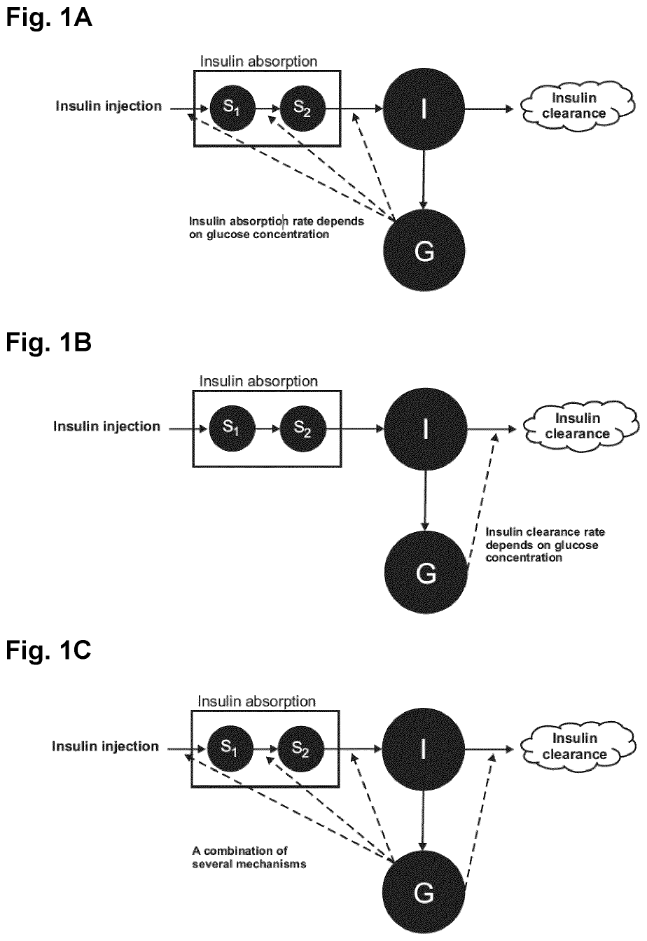

In FIG. 1A-1C examples of how rate constants in a compartment model can be made glucose-dependent by including feedback mechanisms that influence the rate constants are shown. More specifically, FIG. 1A shows a compartment model in which insulin absorption depends on glucose concentration, FIG. 1B shows a compartment model in which insulin clearance depends on glucose concentration, and FIG. 1C shows a compartment model in which insulin clearance depends on a combination of several mechanisms. The compartment model illustrated is an example of one possible model, but the number of compartments and how they interact could be numerous and would depend on the mechanism of the glucose sensitive insulin.

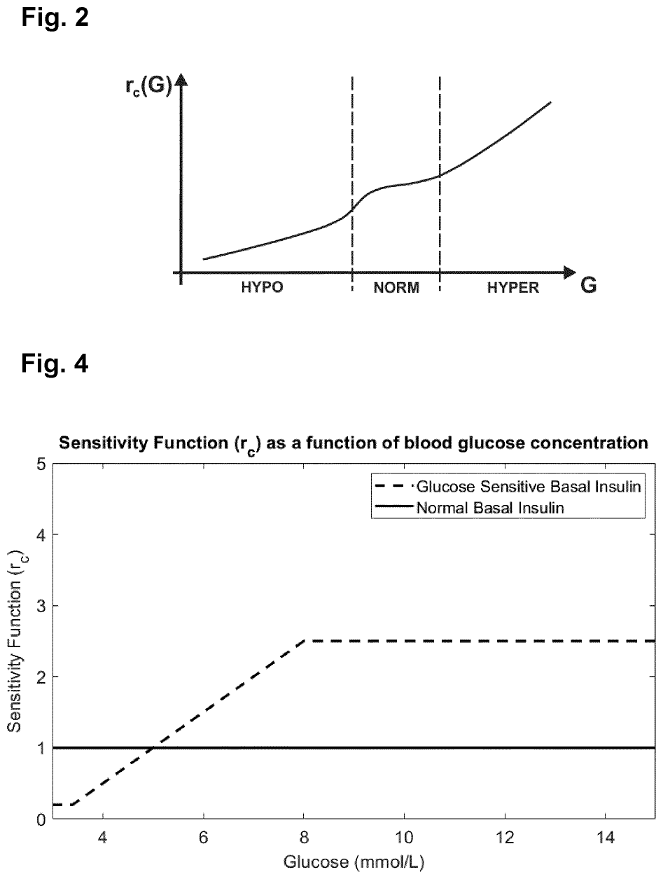

The specific rate constant affected by glucose-concentration depends on the mechanism of the drug. One or several of the rate constants are correlated with the glucose concentration in a linear or nonlinear relationship. This is implemented by letting a rate constant r_{c} be a function of glucose concentration G as shown in FIG. 2. The correlation between the rate constant and the glucose concentration will be drug-specific and is assumed to be known from clinical trials or have been estimated for the individual outside of this algorithm.

Based on continuous glucose measurements and knowledge of previous insulin injections, the algorithm uses an estimator, e.g. a Kalman filter, based on the compartment model to estimate the insulin on board. The estimated IOB at a given point in time is the total amount of insulin in the insulin absorption compartments (there may be one or several), and the amount of insulin in the plasma compartment.

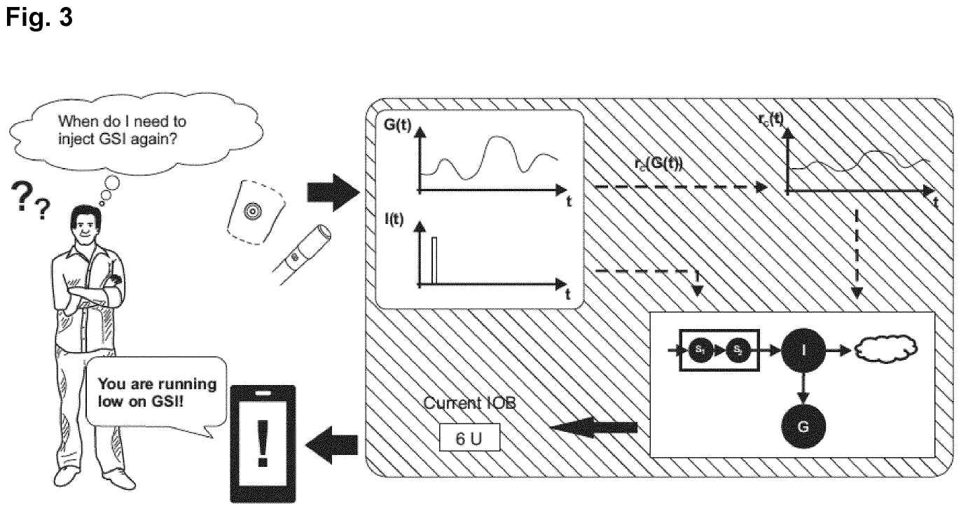

FIG. 3 shows an algorithm overview. The algorithm receives CGM and insulin data as inputs. The changes in one or more rate constants over time are calculated from the CGM readings and the known correlation between glucose-concentration and glucose-dependent rate constants. The rate constants, r_{c}(t), and insulin injection data, I(t), are used as input for an estimator based on a compartment model. The estimator is used to calculate the IOB.

The algorithm outputs the estimated IOB which may be included as part of a decision support tool for GSI users to give feedback and guidance on when to re-inject GSI. The solution could for example be implemented as part of an application on a tablet or smartphone.

In the following an exemplary embodiment of an algorithm implementing aspects of the present invention will be described.

A drug-specific 3 compartment PK model to model the insulin action is used, the model presented in Hovorka et al. to model food compartments and the MVP model presented by Kanderian et al. to model the effect on the blood glucose. Using this combination the insulin on board is estimated.

For IOB computation the following mathematical model (1) of insulin distribution in a subject body is used.

$$\frac{d D e p o t ( t )}{d t} = - {k}_{a} \cdot Depot ( t ) , \qquad ( 1 )$$

$$\frac{d T r a n s i t ( t )}{d t} = {k}_{a} \cdot Depot ( t ) - {k}_{t} \cdot Transit ( t ) ,$$

$$\frac{d C e n t r a l ( t )}{d t} = {k}_{t} \cdot Transit ( t ) - \frac{C L}{V} \cdot Central ( t ) ,$$

$$Depot ( 0 ) = Dose ( 0 ) ,$$

$$Transit ( 0 ) = 0 ,$$

$$Central ( 0 ) = 0$$

The Depot is the injection site (subcutaneous tissue) and Central is the blood pool. Transit does not point to a real physical compartment in the body but rather captures the dynamics of the drug travelling between the two physical compartments (subcutaneous tissue and blood stream).

The model parameters are k_{a,BG} k_{t,BG} [1/min] (the rate constants), CL_{BG} [L/hour] (the rate of insulin clearance from the blood), and V [L] (the volume of insulin distribution in the blood). Dose(0) is the subcutaneous units of insulin injected at time t=0.

The model is an example of a pharmacokinetic model for a GSI insulin. However, in general any other insulin PK model that can represent a given GSI insulin can be used for the concept of the present invention. Examples of alternative mathematical models (2) and (3) for insulin distribution in a subject body is shown below.

$$\frac{dDepot}{dt} = - {k}_{a} * Depot , Depot ( 0 ) = \frac{Dose}{BW} \qquad ( 2 )$$

$$\frac{dCentral}{dt} = {k}_{a} * Depot - \frac{C L}{V} * Central , Central ( 0 ) = 0$$

$$Concentration = \frac{Central}{V}$$

$$\frac{dDepo t ( t )}{dt} = - {k}_{a} * Debot ( t ) , Depot ( 0 ) = Dose \qquad ( 3 )$$

$$\frac{dTransi {t}_{1} ( t )}{dt} = {k}_{a} * Depot ( t ) - {k}_{t} * {Transit}_{1} ( t ) , {Transit}_{1} ( 0 ) = 0$$

$$\frac{dTransi {t}_{2} ( t )}{dt} = {k}_{a} * {Transit}_{1} ( t ) - {k}_{t} * {Transit}_{2} ( t ) , {Transit}_{2} ( 0 ) = 0$$

$$\frac{dTransi {t}_{3} ( t )}{dt} = {k}_{a} * {Transit}_{2} ( t ) - {k}_{t} * {Transit}_{3} ( t ) , {Transit}_{3} ( 0 ) = 0$$

$$\frac{dCentra l ( t )}{dt} = {k}_{t} * {Transit}_{3} ( t ) - \frac{C V}{L} * Central ( t ) , Central ( 0 ) = 0$$

$$Concentration ( t ) = \frac{Central ( t )}{V}$$

The model parameters are parametrized as:

$${k}_{a} = {r}_{{k}_{a}} \cdot {k}_{a , 0} ,$$

$${k}_{t} = {r}_{{k}_{t}} \cdot {k}_{t , 0} ,$$

$$CL = {r}_{c l} \cdot {CL}_{0} .$$

For a non-glucose sensitive insuline, rₖ_{ₐ}=rₖ_{ᵣ}=r_{cl}=1. This denotes that for a non-glucose sensitive insulin the model parameters are fixed at k_{a,0}, k_{t,0}, and CL₀. This would in turn make the PK model linear and thus it would be possible to compute the IOB a priori as a simple time decay. For GSI the IOB cannot be computed as a simple time decay as the ordinary differential equation (ODE) becomes in general non-linear and dependent on how the blood glucose state is modelled. Correspondingly, the ODE may be solved numerically using an estimator (e.g. a Kalman filter) to determine the IOB for GSI's.

For a glucose-sensitive insulin, at least one of the three model parameters will change with blood glucose (BG) concentration via a sensitivity function, r_{c}, according to:

$${r}_{{k}_{a}} = {r}_{c} ( B G ( t ) ) ,$$

$${r}_{{k}_{t}} = {r}_{c} ( B G ( t ) ) ,$$

$${r}_{c l} = {r}_{c} ( B G ( t ) ) .$$

Non-GSI would correspond to r_{c,1}(BG)=r_{c,2}(BG)=r_{c,3}(BG)=1. This would in turn make the PK model linear and thus one would be able to compute the IOB a priori as a simple time decay. For GSI the IOB cannot be computed as a simple time decay as the ordinary differential equation (ODE) becomes in general non-linear and dependent on how the blood glucose state is modelled. Correspondingly, it suggested to solve the ODE numerically and use an estimator (e.g. a Kalman filter) to determine the IOB for GSI's.

As mentioned above, the correlation between the sensitivity function, r_{c}(BG), and the glucose concentration will be drug-specific and is assumed to be known from clinical trials or have been estimated for the individual offline. In this calculation example the sensitivity function as a function of the blood glucose concentration corresponding to FIG. 3 is used. In practice this curve should be estimated for the individual off-line using data.

FIG. 4 presents an exemplary glucose-dependent sensitivity function, r_{c}.

Glucose sensitivity of an insulin can be reached with different methods based on the actual drug design. These different designs result in glucose sensitivity that is functionally different, i.e. through glucose sensitive insulin absorption and/or glucose sensitive clearance of insulin.

For a GSI that has glucose sensitivity in insulin absorption, the rate constants become glucose dependent as follow:

$${k}_{a} = {r}_{{k}_{a}} \cdot {k}_{a , 0} ,$$

$${k}_{t} = {r}_{{k}_{t}} \cdot {k}_{t , 0} ,$$

$${r}_{{k}_{a}} = {r}_{c} ( B G ( t ) ) ,$$

$${r}_{{k}_{t}} = {r}_{c} ( B G ( t ) )$$

The choice of which one (or both) are glucose dependent lies in the design of the drug and which mathematical expression provides the best description.

If the clearance of insulin is glucose sensitive, then the clearance becomes glucose dependent according to

$$C L = {r}_{c l} \cdot {CL}_{0}$$

$${r}_{c l} = {r}_{c} ( B G ( t ) ) .$$

A continuous glucose monitoring (CGM) measures the blood glucose concentration at each time instant t. This means that rₖ_{ₐ}, rₖ_{ₜ}, and r_{cl} can change at each time instant depending on BG level at time t, which consequently makes kₐ, kₜ, and CL time varying parameters. At each time t by solving the model equations we can have an estimate of insulin on board (IOB) computed as:

$$IoB ( t ) = Depot ( t ) + Transit ( t ) + Central ( t ) .$$

Also, we can estimate IOB at a given time horizon of T by numerically solving the model equations in (1) at t−T up to t, i.e., we will have IoB(t−T), IoB(t−T+1), . . . , IoB(t).

For example, assume that a patient injected a U-units dose of GSI at time t=t₀. At time t₀ measurements of the BG concentration using a CGM sensor is started which gives a BG value e.g. every 5 minutes. As time propagates, every 5 minutes a BG measurement is made and therefore an IoB value can be computed using model (1) according to the equation IoB(t)=Depot(t)+Transit(t)+Central(t), with the initial condition of IoB(t₀)=0, Depot(t₀)=0, Transit(t₀)=0, Central(t₀)=0. IoB computation can be continued until a desired time interval, e.g. at time t=t₀+T, the IoB(t₀+T)=Depot(t₀+T)+Transit(t₀+T)+Central(t₀+T). Because the r, function takes a different value at different BG concentrations, the values of r_{c} changes with time if the BG concentration changes over time.

IOB can also be computed retrospectively. At time t_{now} and if it is known that the patient injected a U-unit dose of GSI at T minutes ago, and a CGM sensor measured all the BG concentration values from t_{now}−T up to t_{now}, then solving the equations in model (1) gives an estimate of IOB at t_{now}.

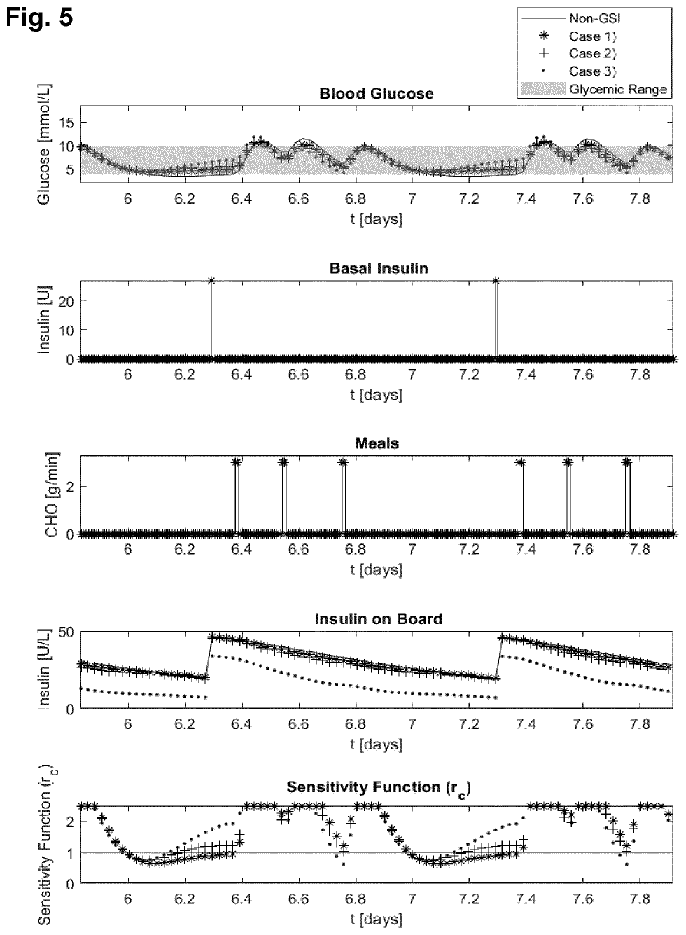

Turning to FIG. 5 three types of GSI insulins are modelled using model (1):

$${r}_{{k}_{a}} = 1 , {r}_{{k}_{t}} = 1 , {r}_{c l} = {r}_{cl} ( B G ( t ) ) \qquad 1 )$$

$${r}_{{k}_{a}} = 1 , {r}_{{k}_{t}} = {r}_{c} ( B G ( t ) ) , {r}_{c l} = {r}_{c} ( B G ( t ) ) \qquad 2 )$$

$${r}_{{k}_{t}} = {r}_{c} ( B G ( t ) ) , {r}_{{k}_{t}} = {r}_{c} ( B G ( t ) ) , {r}_{c l} = {r}_{c} ( B G ( t ) ) \qquad 3 )$$

The first panel shows BG input data from a CGM sensor for each of the three types of insulin as well as for a non-GSI.

A subject with diabetes with the following meal and basal insulin pattern is simulated.
Eats 40 g of CHO at 9.00 every day,
Eats 40 g of CHO at 13.00 every day,
Eats 50 g of CHO at 18.00 every day,
Takes 27 U of basal insulin at 7.00 every day.

This is shown in the third respectively the second panel of FIG. 5. In the present example meal carbohydrates are not used for computing IOB. They have been included merely to have some example values. In a more advanced model meal information can be added to give a more accurate estimate of how the BG will change as a function of time.

The trajectory of the blood glucose, insulin on board and the parameter function as a function of time is plotted as shown in FIG. 5, in which the trajectory of various states for different types of GSI when taking the 27 U of basal insulin is depicted. It can be seen that the parameter that the GSI effects has a large impact on the JOB despite having very similar blood glucose curves. Note that r_{c} takes a different value at each time, t, as the last panel of FIG. 5 indicates. This is because r_{c} depends on BG level and BG level changes with time.

As indicated in FIG. 3 the calculated IOB can be used to adjust the basal insulin dose at injection time. If a given patient were to have eaten a lot of carbohydrates the past day, the insulin on board will be lower at the end of the day than it otherwise would have been if they were to have eaten less carbohydrates. This is because more GSI will have been used and thus less insulin will be left in the body (i.e. lower IOB). As a result a higher dose should be recommended for the next insulin injection.

To use the IOB in an application, e.g. in a bolus calculator when it is desired to adjust the bolus dose by taking into account the basal IOB, a look-up table is provided which gives an IOB value at each BG level at a specific time after the injection of Dose(0). In fact, this is a translation of FIG. 5 into a numerical representation of IOB for practical use. For example, Table 1 indicates the IOB values in three types of GSI cases one hour after the subcutaneous injection of Dose(0)=U units of GSI.

TABLE 1
IOB for different BG values one hour after the single injection
of Dose(0) = U units of GSI. The IOB is represented
as the % of the initial dose, U. The BG concentration at
the time of injection is 5 mmol/L, i.e., BG(t = 0) = 5 mmol/L.

IOB (% of U)

| | Case 1: | Case 2: | Case 3: |
|---|---|---|---|
| Glucose | rₖ_{ₐ} = 1, | rₖ_{ₐ} = 1, | rₖ_{ₜ} = r_{c}(BG(t)), |
| level | rₖ_{ₜ} = 1, | rₖ_{ₜ} = r_{c}(BG(t)), | rₖ_{ₜ} = r_{c}(BG(t)), |
| (mmol/L) | r_{cl} = r_{c}(BG(t)) | r_{cl} = r_{c}(BG(t)) | r_{cl} = r_{c}(BG(t)) |
| 2.0 | 55 | 50 | 10 |
| 2.5 | 56 | 52 | 12 |
| 3.0 | 58 | 53 | 15 |
| 3.5 | 59 | 55 | 17 |
| 4.0 | 60 | 56 | 19 |
| 4.5 | 62 | 58 | 22 |
| 5.0 | 63 | 59 | 24 |
| 5.5 | 64 | 61 | 26 |
| 6.0 | 66 | 63 | 28 |
| 6.5 | 67 | 64 | 31 |
| 7.0 | 68 | 66 | 33 |
| 7.5 | 70 | 67 | 35 |
| 8.0 | 71 | 69 | 38 |
| 8.5 | 73 | 71 | 40 |
| 9.0 | 74 | 72 | 42 |
| 9.5 | 75 | 74 | 45 |
| 10.0 | 77 | 75 | 47 |
| 10.5 | 78 | 77 | 49 |
| 11.0 | 79 | 78 | 52 |
| 11.5 | 81 | 80 | 54 |
| 12.0 | 82 | 82 | 56 |
| 12.5 | 83 | 83 | 58 |
| 13.0 | 85 | 85 | 61 |
| 13.5 | 86 | 86 | 63 |
| 14.0 | 87 | 88 | 65 |
| 14.5 | 89 | 89 | 68 |
| 15.0 | 90 | 91 | 70 |

However, these are only exemplary implementations. For example, aspects of the present invention may be implemented in a sensor device adapted to be mounted e.g. on a skin surface and adapted to measure and log a physiological parameter such as blood glucose values or skin temperatures. Alternatively, the sensor device may be in the form of a device adapted to be implanted, e.g. a pacemaker adapted to measure and log electrocardiographic values.

In the above description of exemplary embodiments, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

1. A method of estimating insulin on board (IOB) for a given glucose sensitive insulin (GSI) in a subject, comprising:
for the given GSI, providing at least one rate constant (r_{c}) as function of glucose concentration (r_{c}(G)),
providing for a period of time a continuous blood glucose log G(t) from the subject,
providing for the period of time an insulin dose log I(t) from the subject,
based on r_{c}(G) and G(t), calculating for each r_{c} a rate constant as function of time (r_{c}(t)), and
based on the at least one r_{c}(t) and I(t) and using an estimating algorithm, calculating an estimated IOB for the subject.

2. A method of estimating IOB as in claim 1, wherein the estimating algorithm is based on a compartment model.

3. A method of estimating IOB as in claim 2, wherein the compartment model comprises at least 2 compartments, at least one transfer rate constant between compartments, and at least one clearance rate constant, at least one of the rate constants being a function of glucose concentration.

4. A computing system for estimating insulin on board (IOB) for a given glucose sensitive insulin (GSI) in a subject, wherein the system comprises one or more processors and a memory, the memory comprising:
instructions that, when executed by the one or more processors, perform a method responsive to receiving a request for calculation of an IOB value, the method comprising:
for the given GSI, providing at least one rate constant (r_{c}) as function of glucose concentration (r_{c}(G)),
providing for a period of time a continuous blood glucose log G(t) from the subject,
providing for the period of time an insulin dose log I(t) from the subject,
based on r_{c}(G) and G(t), calculating for each r_{c} a rate constant as function of time (r_{c}(t)), and
based on the at least one r_{c}(t) and I(t) and using an estimating algorithm, calculating IOB for the subject.

5. A computing system as in claim 4, wherein the estimating algorithm is based on a compartment model.

6. A computing system as in claim 5, wherein the compartment model comprises at least 2 compartments, at least one transfer rate constant between compartments, and at least one clearance rate constant, at least one of the rate constants being a function of glucose concentration.

7. A computing system as in claim 4, adapted to provide a long-acting or ultra-long-acting insulin dose recommendation (ADR) for a subject to treat diabetes mellitus, the memory further comprising:
instructions that, when executed by the one or more processors, perform a method responsive to receiving a dose guidance request (DGR), the method further comprising:
providing the long-acting or ultra-long-acting insulin ADR, the recommendation being calculated based on the estimated IOB.
